(19) United States
(12) Patent Application Publication
(10) Pub. No.: US 2007/0287688 A1
(43) Pub. Date: Dec. 13, 2007

(54) Pharmaceutical Compositions for Topical Application

(75) Inventors: Thomas Chan, Hopkinton, MA (US); Scott Krauser, Lexington, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Macro Chem Corporation

(21) Appl. No.: 10/564,199

(22) PCT Filed: Jul. 9, 2004
(86) PCT No.: PCT/US04/21855

§ 371 Date: Sep. 14, 2006

Related U.S. Application Data
(60) Provisional application No. 60/486,236, filed on Jul. 11, 2003.

(51) Int. Cl. A61K 31/05; A61K 31/19; A61K 31/195; A61K 31/20; A61K 31/4965; A61K 31/56; A61K 47/00; A61P 17/00

(52) U.S. Cl. 514/169; 514/255.05; 514/558; 514/563; 514/568; 514/573; 514/731; 514/784

(57) ABSTRACT

Compounds of Formula I are useful as skin penetration enhancing agents: (I) wherein: R represents a linear, saturated or unsaturated, substituted or unsubstituted hydrocarbyl radical; and X represents a —(CO)O—, —O(CO)—, C═O, CONH, O, NHCONH, S, or S═O radical. Compositions and methods using these compounds are also described.

### CROSS REFERENCE TO RELATED APPLICATION

This application claims the benefit of priority from U.S. Provisional Ser. No. 60/486,236, filed Jul. 11, 2003, the entirety of which is incorporated herein by reference.

### FIELD OF THE INVENTION

The present invention relates generally to pharmaceutical compositions for administration of active agents through the skin and other membranes and methods of preparing and using the same.

Recently, transdermal therapeutic formulations have been developed to deliver active agents to the body via the skin or other membranes, e.g., mucosa. These formulations offer the advantages of allowing the active agents to evade metabolism in the intestine and liver, reduce side reactions and provide a longer pharmacological effect. However, their use has been limited because skin naturally provides a barrier to foreign substances, such as most active agents. Therefore, only limited kinds of active agents can attain effective concentrations in skin tissues and within the bloodstream.

Various attempts have been made to overcome these problems. One approach has been to increase percutaneous absorption of active agents by decreasing the barrier property of skin through the use of skin penetration enhancing (SPE) compounds. However, problems of compatibility of SPE compounds with the active agents and/or carriers, or with the efficacy of the enhancers themselves continue to occur. In addition, skin irritation and other systemic and local side effects have proven to be problematic. Further improvements are still needed to overcome these problems.

One embodiment of the present invention provides a pharmaceutical composition comprising:

a) at least one active agent; and

b) a skin penetration enhancer represented by the following Formula I:

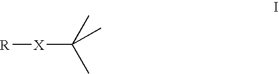

wherein:

R represents a linear, saturated or unsaturated, substituted or unsubstituted hydrocarbyl radical; and

X represents a —(CO)O—, —O(CO)—, >C═O, —CONH—, —O—, —NHCONH—, —S—, or >S═O radical.

One embodiment of the present invention provides a pharmaceutical composition comprising:

a) at least one active agent; and

b) a skin penetration enhancer represented by the Formulas IA or IB

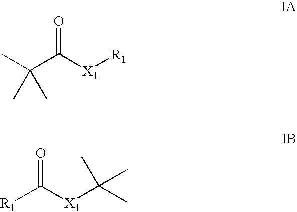

wherein:

R₁ represents a linear or branched, saturated or unsaturated, substituted or unsubstituted hydrocarbyl radical; and

X₁ is either an oxygen atom or an NH radical.

Another embodiment of the present invention provides a pharmaceutical composition comprising:

a) from about 1 wt. % to about 15 wt. % buspirone hydrochloride; and

b) a skin penetration enhancer represented by the Formulas IA or IB

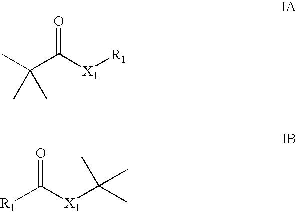

wherein:

R₁ represents a linear or branched, saturated or unsaturated, substituted or unsubstituted hydrocarbyl radical; and

X₁ is either an oxygen atom or an NH radical.

Another embodiment of the present invention provides a pharmaceutical composition comprising:

a) from about 1 wt. % to about 10 wt. % ibuprofen; and

b) a skin penetration enhancer represented by the Formulas IA or IB

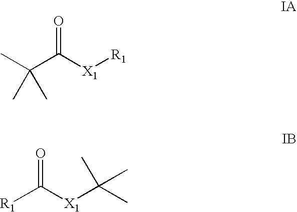

wherein:

R₁ represents a linear or branched, saturated or unsaturated, substituted or unsubstituted hydrocarbyl radical; and

X₁ is either an oxygen atom or an NH radical.

Another embodiment of the present invention provides a pharmaceutical composition comprising:

a) from about 0.5 wt. % to about 5 wt. % testosterone; and b) a skin penetration enhancer represented by the Formulas IA or IB

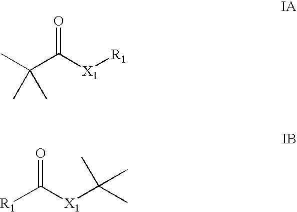

wherein:

R₁ represents a linear or branched, saturated or unsaturated, substituted or unsubstituted hydrocarbyl radical; and

X₁ is either an oxygen atom or an NH radical.

Another embodiment of the present invention provides a pharmaceutical composition comprising:

a) from about 0.5 wt. % to about 5 wt. % PGE-1; and

b) a skin penetration enhancer represented by the Formulas IA or IB

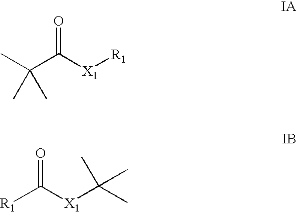

wherein:

R₁ represents a linear or branched, saturated or unsaturated, substituted or unsubstituted hydrocarbyl radical; and

X₁ is either an oxygen atom or an NH radical.

Another embodiment of the present invention provides a pharmaceutical composition comprising:

a) from about 1.5 wt. % to about 3.5 wt. % hydroquinone; and

b) a skin penetration enhancer represented by the Formulas IA or IB

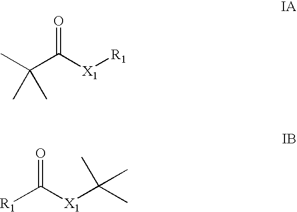

wherein:

R₁ represents a linear or branched, saturated or unsaturated, substituted or unsubstituted hydrocarbyl radical; and

X₁ is either an oxygen atom or an NH radical.

Another embodiment of the present invention provides a method for forming a pharmaceutical composition comprising, mixing:

a) from about 1 wt. % to about 10 wt. % of an active agent

b) a skin penetration enhancer represented by the Formulas IA or IB

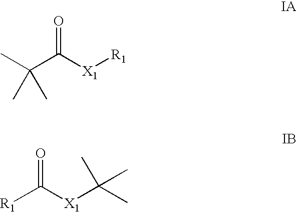

wherein:

R₁ represents a linear or branched, saturated or unsaturated, substituted or unsubstituted hydrocarbyl radical; and

X₁ is either an oxygen atom or an NH radical.

Another embodiment of the present invention provides a method of administering an active agent to an animal or plant in need thereof comprising topically applying to said animal or plant a pharmaceutical composition comprising:

a) an active agent; and

b) a skin penetration enhancer represented by the Formulas IA or IB

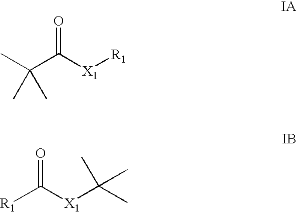

wherein:

R₁ represents a linear or branched, saturated or unsaturated, substituted or unsubstituted hydrocarbyl radical; and

X₁ is either an oxygen atom or an NH radical.

### BRIEF DESCRIPTION OF THE DRAWINGS

FIG. 1 is a graph showing the measured flux of ibuprofen as a function of time across human skin when applied as a 5% solution of ibuprofen in ethanol alone (14), or as an ethanolic solution containing 10% N-decyl pivalamide (34), 10% N-dodecyl pivalamide (35), 10% tetradecyl pivalate (33), 10% t-butyl laurate (10), 10% lauryl pivalate (13), 10% t-butyl decanoate (22), or 10% t-butyl myristoate (23). Parenthetical numbers are in reference to the solutions reported in Table 1.

FIG. 2 is a graph showing cumulative transfer of ibuprofen across human skin as a function of time when applied as a 5% solution of ibuprofen in ethanol alone (14), or as an ethanolic solution containing 10% N-decyl pivalamide (34), 10% N-dodecyl pivalamide (35), 10% tetradecyl pivalate (33), 10% t-butyl laurate (10), 10% lauryl pivalate (13), 10% t-butyl decanoate (22), or 10% t-butyl myristoate (23). Parenthetical numbers are in reference to the solutions reported in Table 1.

FIG. 3 is a graph showing the measured flux of PGE-1 as a function of time across human skin when applied as a 2% solution of PGE-1 in ethanol alone, or as an ethanolic solution containing 10% t-butyl decanoate (25), 10% t-butyl myristoate (26), 10% t-butyl laurate (27), 10% lauryl pivalate (28), or 10% tetra decyl pivalate (47). Parenthetical numbers are in reference to the solutions reported in Table 1.

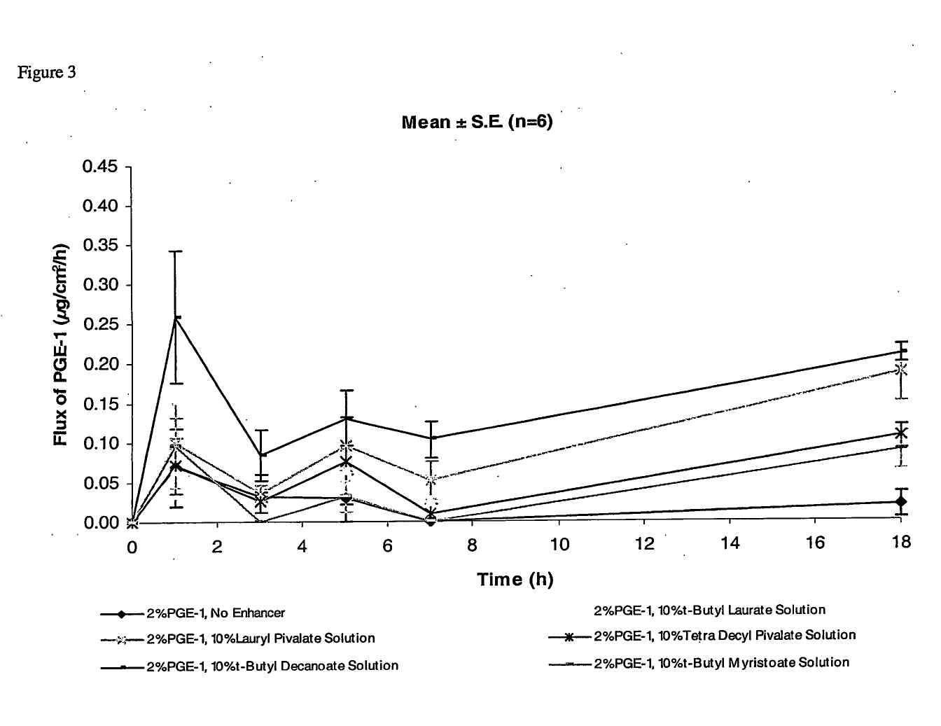

FIG. 4 is a graph showing cumulative transfer of PGE-1 across human skin as a function of time when applied as a 2% solution of PGE-1 in ethanol alone, or as an ethanolic solution containing 10% t-butyl decanoate (25), 10% t-butyl myristoate (26), 10% t-butyl laurate (27), 10% lauryl pivalate (28), or 10% tetra decyl pivalate (47). Parenthetical numbers are in reference to the solutions reported in Table 1.

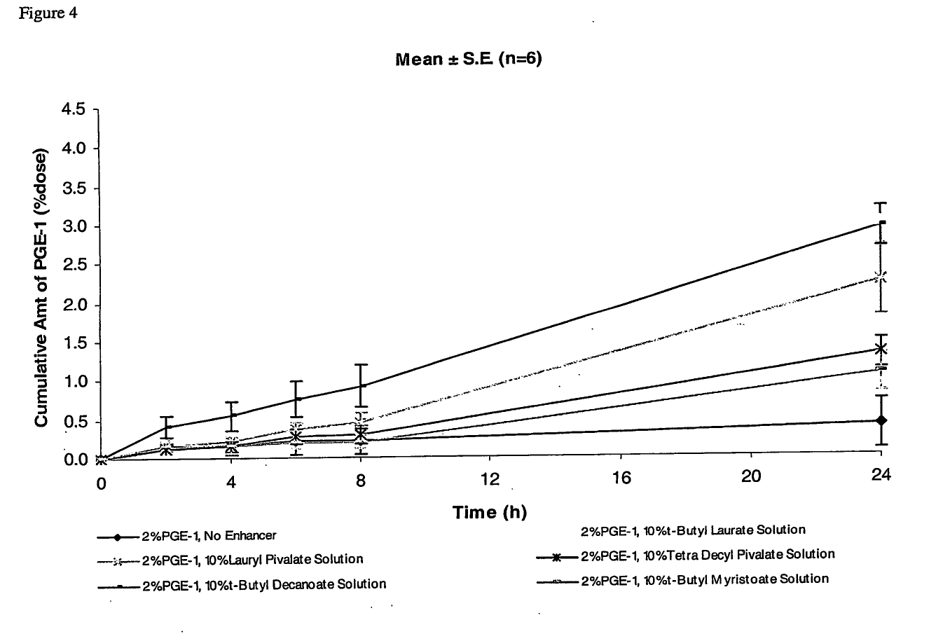

FIG. 5 is a graph showing the measured flux of PGE-1 as a function of time across human skin when applied as a 2% solution of PGE-1 in ethanol alone, or as an ethanolic solution containing 10% N-decyl pivalamide (48), or 10% N-dodecyl pivalamide (49). Parenthetical numbers are in reference to the solutions reported in Table 1.

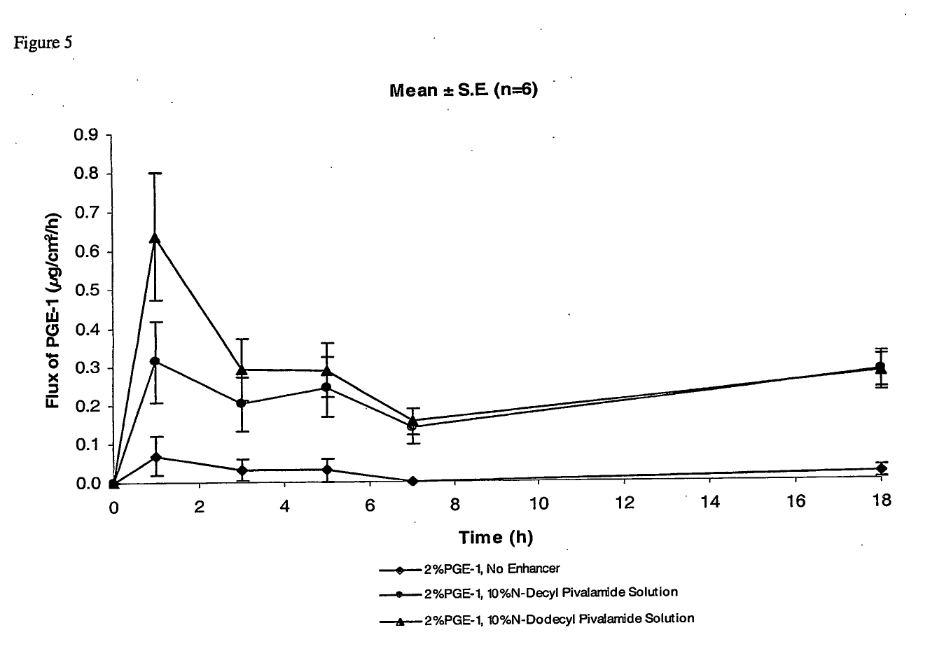

FIG. 6 is a graph showing cumulative transfer of PGE-1 across human skin as a function of time when applied as a 2% solution of PGE-1 in ethanol alone, or as an ethanolic solution containing 10% N-decyl pivalamide (48), or 10% N-dodecyl pivalamide (49). Parenthetical numbers are in reference to the solutions reported in Table 1.

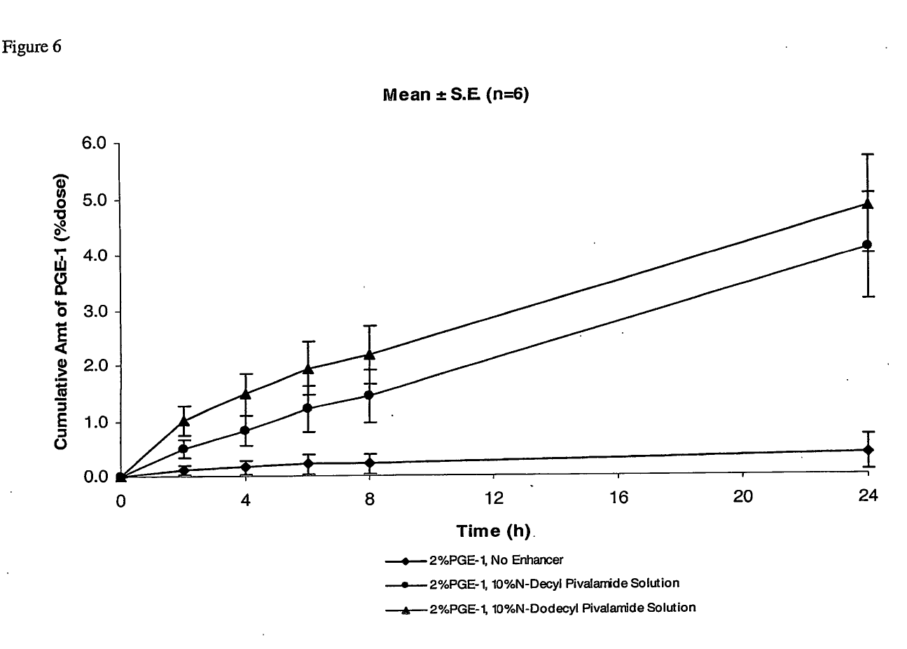

FIG. 7 is a graph showing the measured flux of testosterone as a function of time across human skin when applied as a 1% solution of testosterone in ethanol alone (7), or as an ethanolic solution containing 10% lauryl pivalate (6). Parenthetical numbers are in reference to the solutions reported in Table 1.

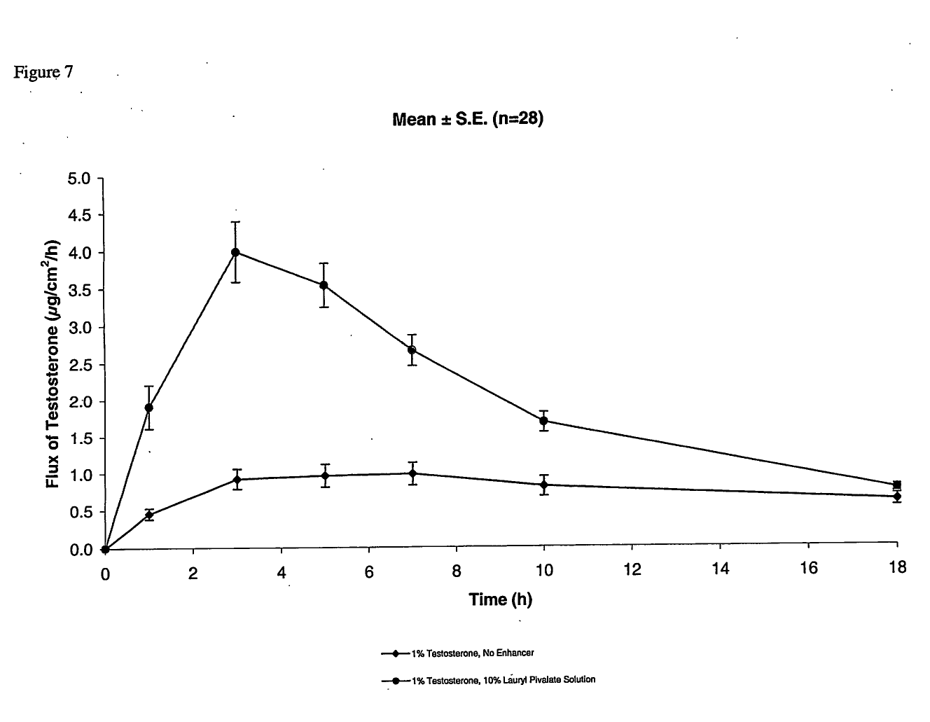

FIG. 8 is a graph showing cumulative transfer of testosterone across human skin as a function of time when applied as a 1% solution of testosterone in ethanol alone (7), or as an ethanolic solution containing 10% lauryl pivalate (6). Parenthetical numbers are in reference to the solutions reported in Table 1.

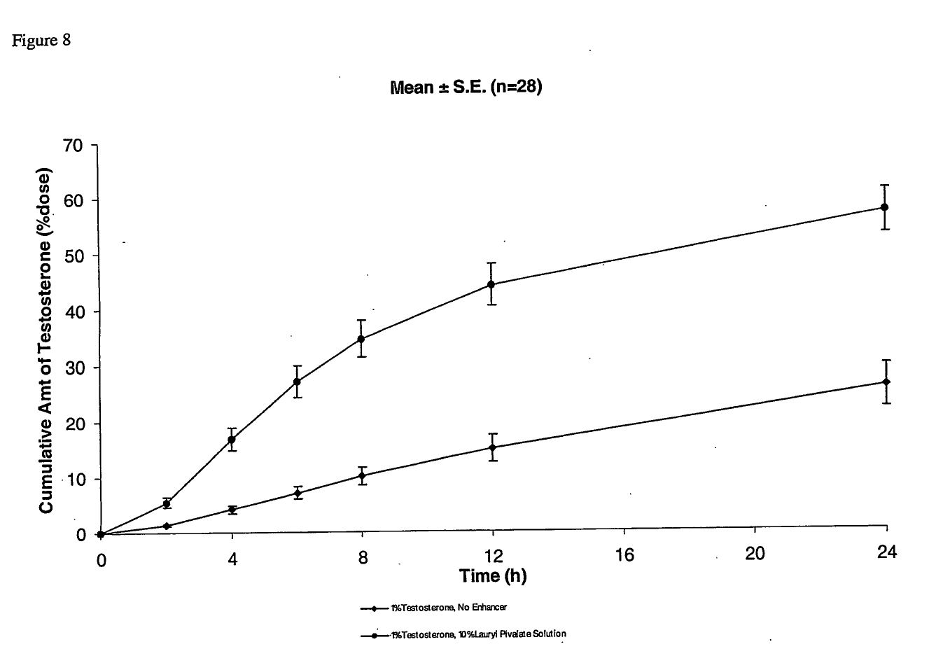

FIG. 9 is a graph showing the measured flux of testosterone as a function of time across human skin when applied as a 1% solution of testosterone in ethanol alone (7), or as an ethanolic solution containing 10% t-butyl myristoate (24), 10% N-decyl pivalamide (38), or as a 10% N-dodecyl pivalamide (39). Parenthetical numbers are in reference to the solutions reported in Table 1.

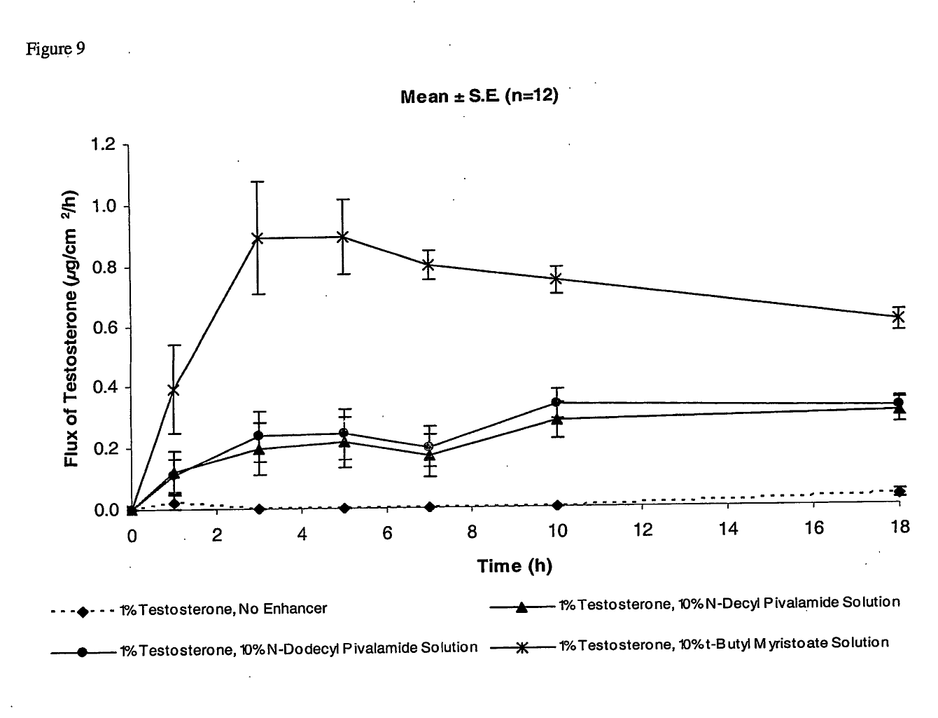

FIG. 10 is a graph showing cumulative transfer of testosterone across human skin as a function of time when applied as a 1% solution of testosterone in ethanol alone (7), or as an ethanolic solution containing 10% t-butyl myristoate (24), 10% N-decyl pivalamide (38), or as a 10% N-dodecyl pivalamide (39). Parenthetical numbers are in reference to the solutions reported in Table 1.

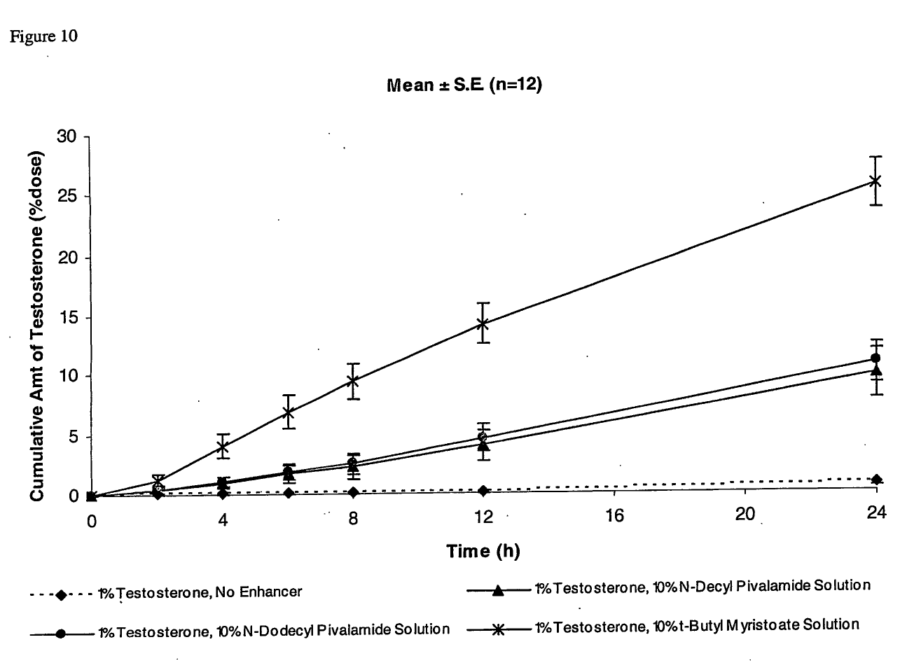

FIG. 11 is a graph showing the measured flux of testosterone as a function of time across human skin when applied as a 1% solution of testosterone in ethanol alone (7), or as an ethanolic solution containing 10% t-butyl decanoate (40). Parenthetical numbers are in reference to the solutions reported in Table 1.

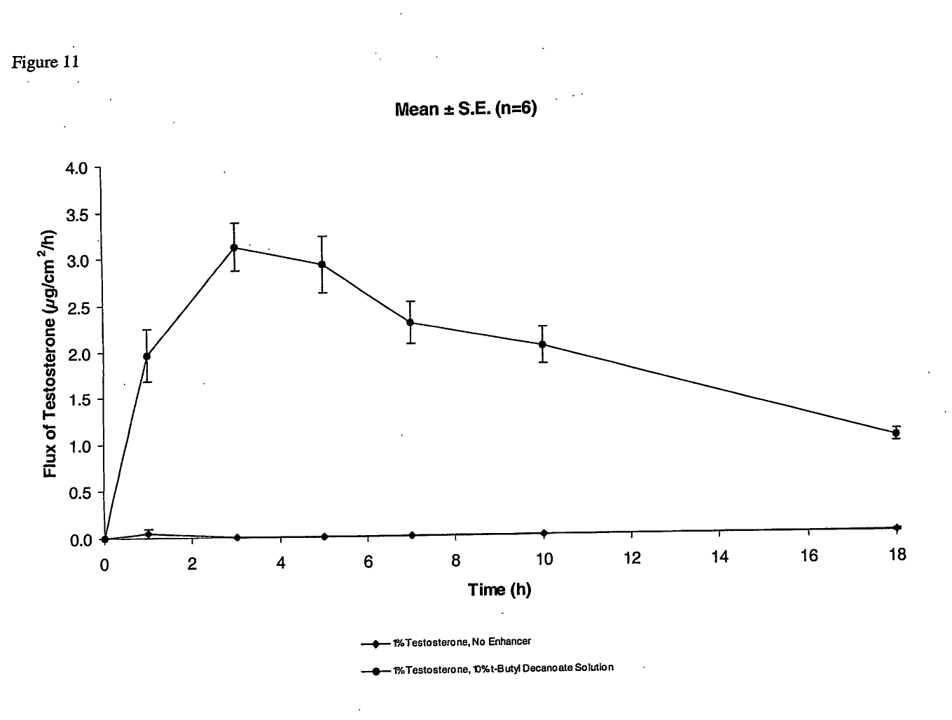

FIG. 12 is a graph showing cumulative transfer of testosterone across human skin as a function of time when applied as a 1% solution of testosterone in ethanol alone (7), or as an ethanolic solution containing 10% t-butyl decanoate (40). Parenthetical numbers are in reference to the solutions reported in Table 1.

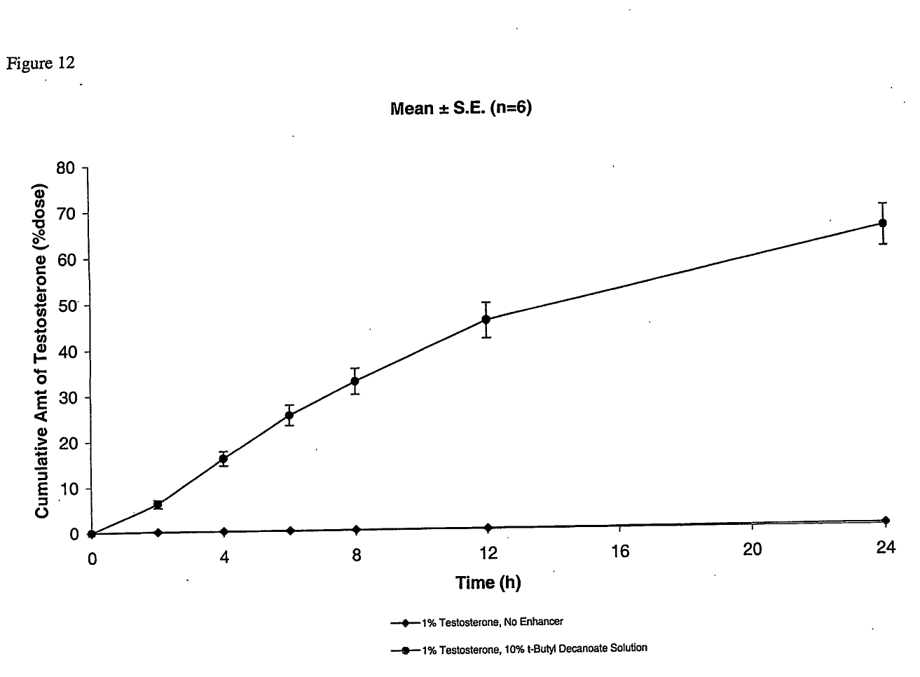

FIG. 13 is a graph showing the measured flux of buspirone as a function of time across human skin when applied as a 10% solution of buspirone in aqueous ethanol (52), or as an ethanolic solution containing 10% N-decyl pivalamide (61), 10% N-dodecyl pivalamide (69), 10% t-butyl laurate (66), 10% lauryl pivalate (50), 10% t-butyl decanoate (51), or 10% t-butyl myristoate (63). Parenthetical numbers are in reference to the solutions reported in Table 1.

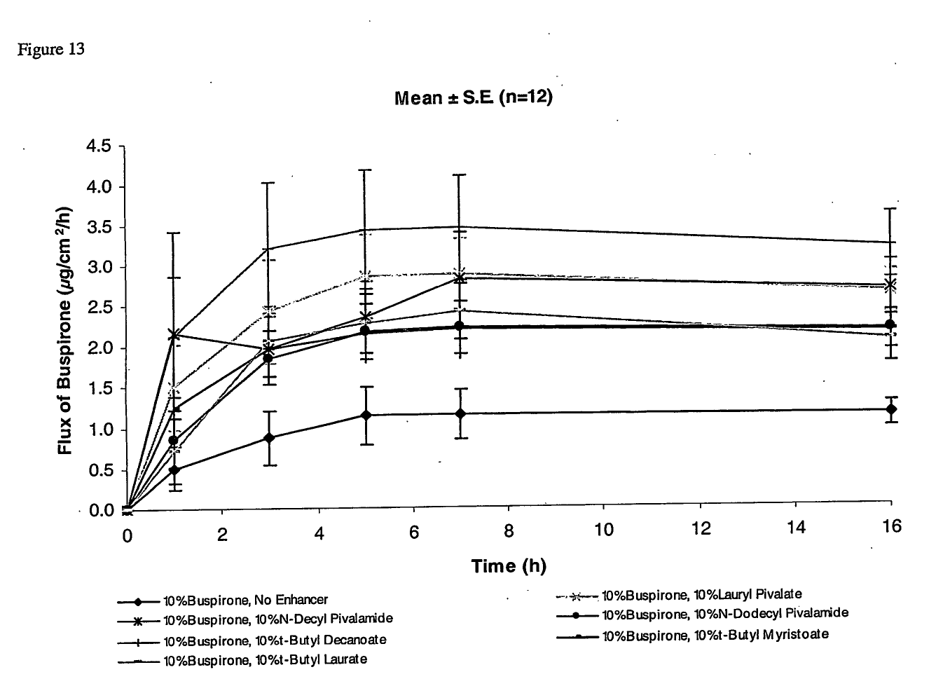

FIG. 14 is a graph showing the cumulative transfer of buspirone as a function of time across human skin when applied as a 10% solution of buspirone in aqueous ethanol (52), or as an ethanolic solution containing 10% N-decyl pivalamide (61), 10% N-dodecyl pivalamide (69), 10% t-butyl laurate (66), 10% lauryl pivalate (50), 10% t-butyl decanoate (51), or 10% t-butyl myristoate (63). Parenthetical numbers are in reference to the solutions reported in Table 1.

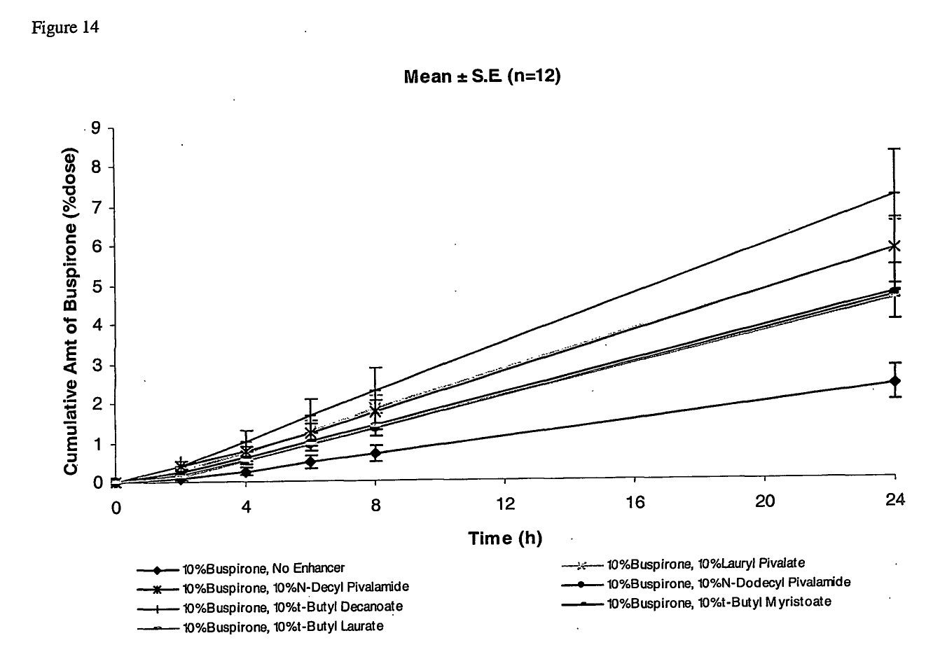

FIG. 15 is a graph showing the measured flux of hydroquinone as a function of time across human skin when applied as a 3% solution of hydroquinone in ethanol alone (30), or as an ethanolic solution containing 10% N-decyl pivalamide (56), 10% N-dodecyl pivalamide (57), 10% t-butyl laurate (31), 10% lauryl pivalate (29), 10% t-butyl decanoate (58), 10% t-butyl myristoate (59), or 10% tetradecyl pivalate (55). Parenthetical numbers are in reference to the solutions reported in Table 1.

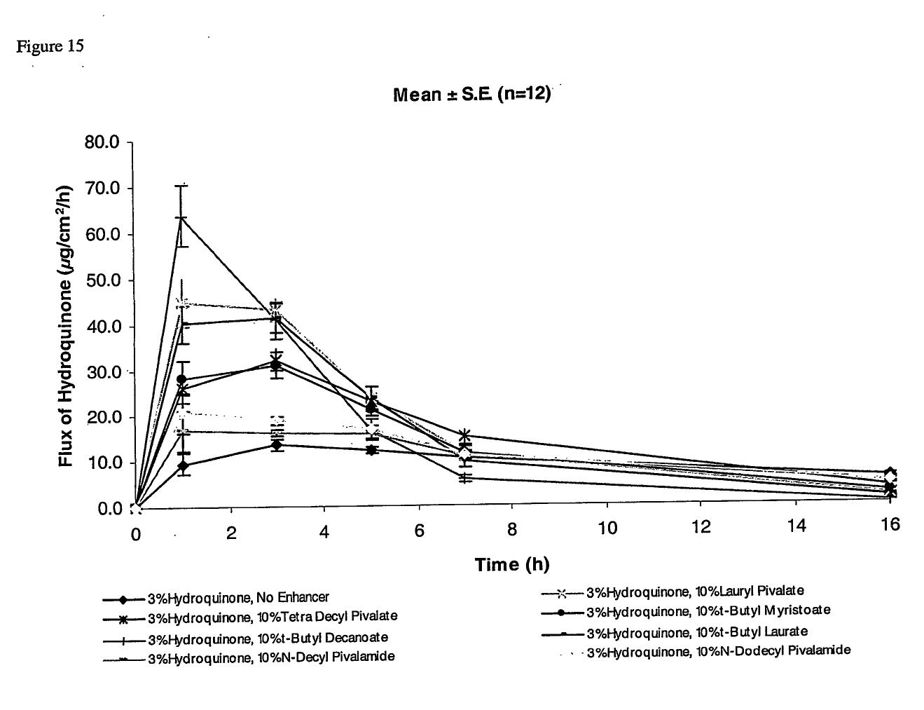

FIG. 16 is a graph showing the cumulative transfer of Hydroquinone as a function of time across human skin when applied as a 3% solution of Hydroquinone in ethanol alone (30), or as an ethanolic solution containing 10% N-decyl pivalamide (56), 10% N-dodecyl pivalamide (57), 10% t-butyl laurate (31), 10% lauryl pivalate (29), 10% t-butyl decanoate (58), 10% t-butyl myristoate (59), or 10% tetradecyl pivalate (55). Parenthetical numbers are in reference to the solutions reported in Table 1.

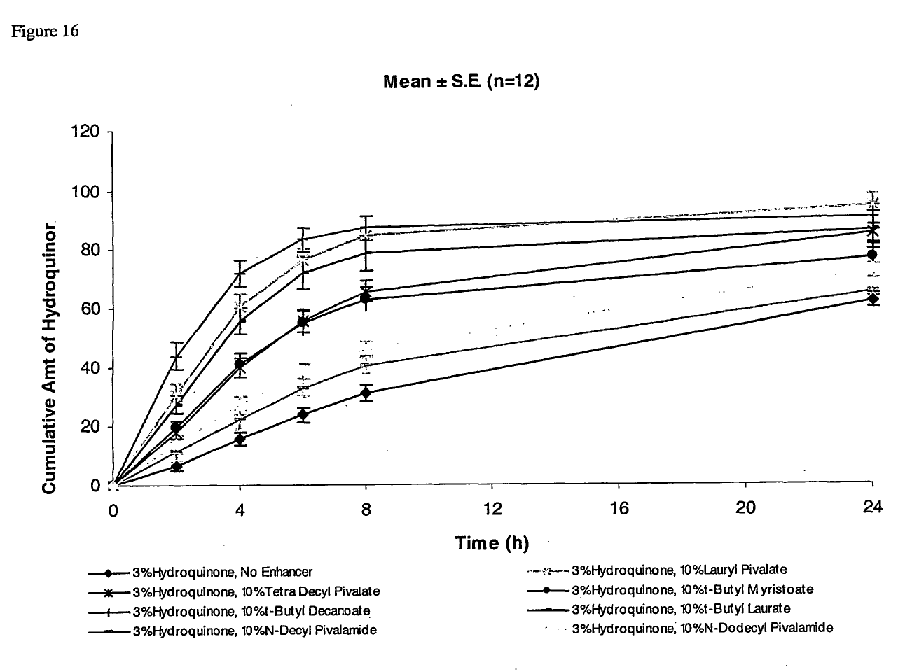

FIG. 17 is a graph showing the measured flux of PGE-1 as a function of time across human skin when applied as a 1% solution of PGE-1 in ethanol alone (21), or as an ethanolic solution containing 10% N-decyl pivalamide (43), 10% N-dodecyl pivalamide (44), 10% t-butyl laurate (17), 10% lauryl pivalate (20), 10% t-butyl decanoate (53), 10% t-butyl myristoate (54), or 10% tetradecyl pivalate (42). Parenthetical numbers are in reference to the solutions reported in Table 1.

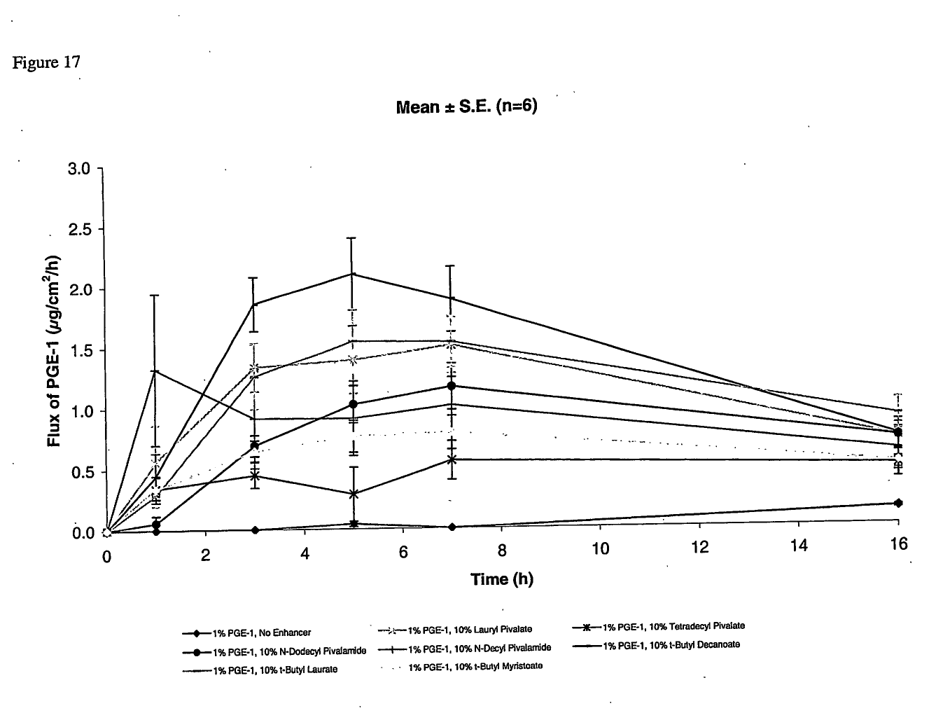

FIG. 18 is a graph showing the cumulative transfer of PGE-1 as a function of time across human skin when applied as a 1% solution of PGE-1 in ethanol alone (21), or as an ethanolic solution containing 10% N-decyl pivalamide (43), 10% N-dodecyl pivalamide (44), 10% t-butyl laurate (17), 10% lauryl pivalate (20), 10% t-butyl decanoate (53), 10% t-butyl myristoate (54), or 10% tetradecyl pivalate (42). Parenthetical numbers are in reference to the solutions reported in Table 1.

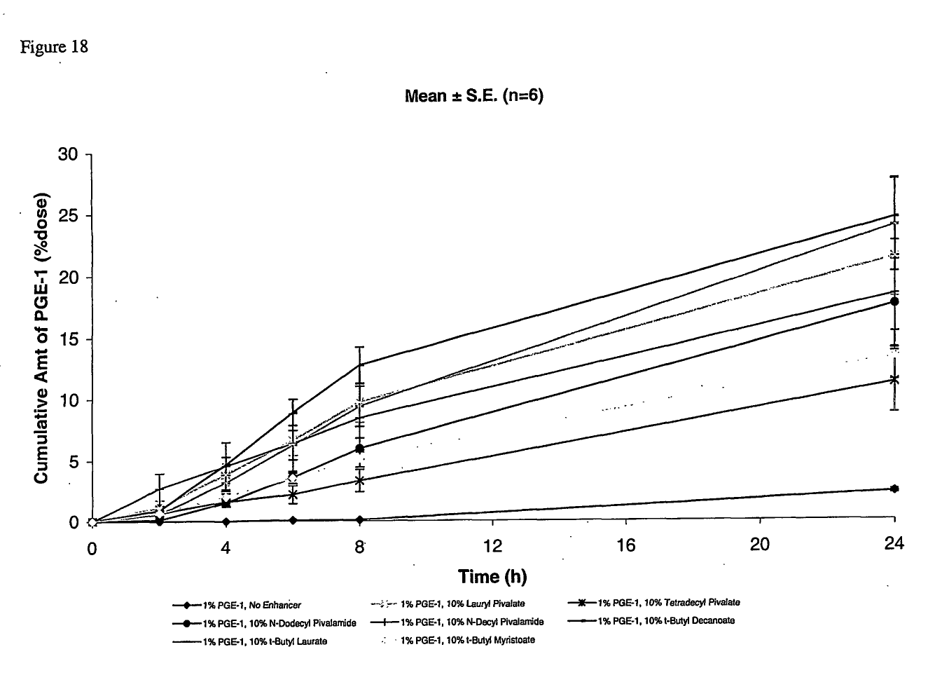

As used herein the following terms have the following meanings:

“pharmaceutically acceptable” refers to substances that, when taking into account the benefits versus the risks, are acceptable for use with mammals, including humans, without undue adverse side effects (such as toxicity, irritation, and allergic response).

Skin Penetration Enhancers

In one embodiment, skin penetration enhancing compounds of the present invention may be represented by the following Formula I: 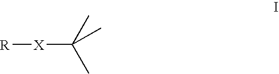
wherein: R represents a linear, saturated or unsaturated, substituted or unsubstituted hydrocarbyl radical; and X represents a —(CO)O—, —O(CO)—, >C═O, —CONH—, —O—, —NHCONH—, —S—, >S═O radical. Non-limiting examples of compounds of Formula I include, esters, amides, ketones, ethers, urethanes, thioethers and sulfoxides.

In one embodiment of the present invention, skin penetration enhancer compounds of Formula I may be selected from compounds represented by Formulas IA or IB: 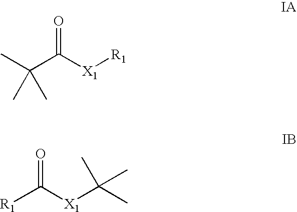
wherein: R₁ represents a linear, saturated or unsaturated, substituted or unsubstituted hydrocarbyl radical; and X₁ is either an oxygen atom or an NH radical.

In one embodiment of the invention the compounds of Formulas IA and IB include those where R₁ represents a linear C6-C20 alkyl radical, for example, a C6-C16 alkyl radical, a C6-14 alkyl, or a C8-C14 alkyl radical. R₁ may, for example, represent a C8-C14 linear alkyl radical, for example, an octyl, nonyl, decyl, undecyl, dodecyl, tridecyl, or tetradecyl radical. In one embodiment, R₁ represents a linear alkyl radical with an even number of carbon atoms, for example, an octyl, decyl, dodecyl or tetradecyl radical.

Exemplary skin penetration enhancer compounds of Formulas IA and IB include decyl pivalate, dodecyl pivalate, tetradecyl pivalate, N-decyl pivalamide, N-dodecyl pivalamide, tert-butyl decanoate, tert-butyl laurate, and tert-butyl myristoate.

Often compounds of Formulas IA or IB generally provide less odor than other enhancers and may be more stable in formulations containing them.

The enhancers of the present invention may often be present from about 1 wt. % or 2 wt. % to about 15 wt. % or 20 wt. %, based on the total weight of this composition, for example, about 1 wt. %, 2 wt. %, 3 wt. %, 4 wt. %, 5 wt. %, 6 wt. %, 7 wt. %, 8 wt. %, 9 wt. %. Higher or lower amounts may also be effective depending on, for example, the active agent, the depth and rate of penetration desired, and the type of other ingredients present.

Compounds according to Formulas IA and IB may be synthesized by techniques known in the art. For example, esters of these Formulas can be synthesized by an esterification reaction between the constituent alcohol and acid. See, for example, Wiener and Gilon, J. Mol. Catalysis 37: 45-52, 1986.

The skin penetration enhancing compounds of Formulas IA or IB may be used individually or in combination with each other or in admixture with other known skin penetration enhancing compounds, such as those described below.

Known skin penetration enhancing compounds which may be used in combination with the compound(s) of Formula IA and/or IB include, for example:
(i) 2-substituted 1,3-dioxolanes of the formula (I): 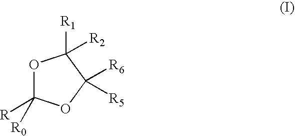
   or a 2-substituted 1,3-dioxane of the formula (II): 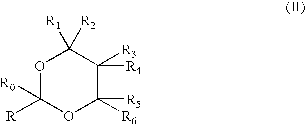
   or an acetal (including hemiacetal) of the formula (III): 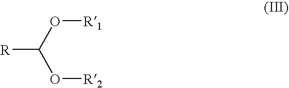
   where R represents a C6 to C20 aliphatic group, R₀, R₁, R₂, R₃, R₄, R₅, and R₆, each, independently, represent hydrogen or a C1 to C4 aliphatic group; R′₁ and R′₂, each, independently, may represent a hydrogen or a C1 to C4 aliphatic group, with the proviso that both R′₁ and R′₂ do not simultaneously represent hydrogen. Compounds of these formulas may be available commercially from MacroChem Corporation under the trademark SEPA□.

R may also represent a C6 to C12 aliphatic group; especially C7 to C10 aliphatic group. The aliphatic group may be a straight or branched chain alkyl or alkenyl group, such as, for example, n-hexyl, n-heptyl, n-octyl, n-nonyl, n-decyl, n-undecyl, n-dodecyl, n-hexadecyl, n-octadecyl, 2-methyl-octyl, 4-ethyl-decyl, 8-methyl-decyl, n-octenyl, n-stearyl, and the like.

The C1 to C4 aliphatic group may be, for example, methyl, ethyl, n-propyl, isopropyl, n-butyl, tert-butyl, ethenyl, and the like. For example, R₁ to R₆ may represent aliphatic groups and R′₁ and R′₂ may represent alkyl groups, for example, alkyls having 1 or 2 carbon atoms, such as ethyl. R₁ to R₆ may also all be hydrogen.

Representative skin penetration enhancing compounds of formulas (I), (II) and (III) include, for example, 2-n-heptyl-1,3-dioxolane, 2-n-nonyl-1,3-dioxolane, 2-n-undecyl-1,3-dioxolane, 2-n-nonyl-1,3-dioxane, 2-n-undecyl-1,3-dioxane, 2-n-heptylaldehyde-acetal, 2-n-octyl-aldehyde-acetal, 2-n-nonylaldehyde-acetal, 2-n-decylaldehyde-acetal, 3,7-dimethyl-2,6-octadienal (citral), citronal and the like. See also U.S. Pat. Nos. 5,942,545 and 5,976,566.

Another class of skin penetration enhancing compounds (ii) are cyclic ketones and cyclic lactones and derivatives thereof, as disclosed in, for example, U.S. Pat. Nos. 5,023,252 and 5,731,303, the disclosures of which, are incorporated herein, in their entireties, by reference thereto.

The skin penetration enhancing compounds (II) may be represented by the following formula (IV): 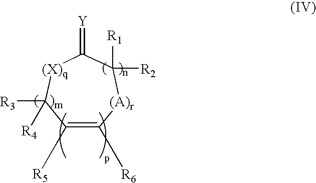
wherein X and Y are oxygen, sulfur or an imino group of the structure 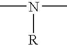
or ═N—R, with the proviso that when Y is the imino group, X is an imino group, and when Y is sulfur, X is sulfur or an imino group, A is group having the structure 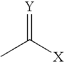
wherein X and Y are defined above,

m and n are integers having a value from 1 to 20 and the sum of m+n is not greater than 25,

p is an integer having a value of 0 or 1,

q is an integer having a value of 0 or 1,

r is an integer having a value of 0 or 1,

R represents hydrogen or a straight or branched chain alkyl group having from 1 to 6 carbon atoms, and,

R₁, R₂, R₃, R₄, R₅ and R₆, each, independently, represent hydrogen or a straight or branched chain alkyl group having from 1 to 6 carbon atoms, with the proviso that only one of R₁ to R₆ may be said alkyl group, and with the further provisos that,

when p, q and r have a value of 0 and Y is oxygen, m+n is at least 11,

when X is an imino group, q equals 1, Y is oxygen, and p and r are 0, then m+n is at least 11.

Examples of the alkyl group for R and R1 to R6 include methyl, ethyl, n-propyl, isopropyl, n-butyl, isobutyl, sec-butyl, amyl, hexyl, and the like.

For example, each of R and R₁ to R₆ may represent hydrogen atoms and X and Y may each represent oxygen. Compounds represented by formula (IV) may be cyclic ketones (when q and r are each 0) or cyclic lactones.

Other compounds of formula (IV) may be represented by the following general formula (IV-A): 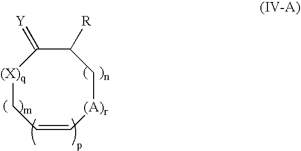
wherein X, Y, R, A, m, n, p, q and r, are as defined above.

For example, in formula (IV-A), X and Y may each represent oxygen and R may represent hydrogen.

For example, pentadecalactone is a skin penetration enhancer of type (ii).

Another class of skin penetration enhancing compounds (iii) include an alkyl 2 (N,N disubstituted amino)alkanoate, an (N,N disubstituted amino)alkanol alkanoate, or a mixture of these, as more fully described in U.S. Pat. No. 6,046,244, the disclosure of which is incorporated herein by reference thereto. For convenient reference, alkyl 2 (N,N disubstituted amino)alkanoates and (N,N disubstituted amino)alkanol alkanoates can be grouped together under the label alkyl(N,N disubstituted amino)esters.

Alkyl 2 (N,N disubstituted amino)alkanoates useful as skin penetration enhancers may also be represented by the following formula (V)

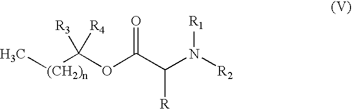

wherein n is an integer having a value in the range of about 4 to about 18;

R is a member of the group consisting of hydrogen, C1 to C7 alkyl, benzyl and phenyl;

R₁ and R₂ are members of the group consisting of hydrogen and

C1 to C7 alkyl; and R₃ and R₄ are members of the group consisting of hydrogen, methyl and ethyl.

Exemplary alkyl(N,N disubstituted amino)alkanoates include C4 to C18 alkyl (N,N disubstituted amino)acetates and C4 to C18 alkyl(N,N disubstituted amino)propionates. Exemplary specific alkyl 2 (N,N disubstituted amino)alkanoates include dodecyl 2 (N,N dimethylamino)propionate (DDAIP); and dodecyl 2 (N,N dimethylamino)acetate (DDAA).

Alkyl 2 (N,N disubstituted amino)alkanoates are known. For example, dodecyl 2 (N,N dimethylamino)propionate (DDAIP) is available from Steroids, Ltd. (Chicago, Ill.). In addition, alkyl 2 (N,N disubstituted amino)alkanoates can be synthesized from more readily available compounds as described in U.S. Pat. No. 4,980,378 to Wong et al., which syntheses procedures are incorporated herein by reference.

Suitable (N,N-disubstituted amino)-alkanol alkanoates can be represented by the formula (VI):

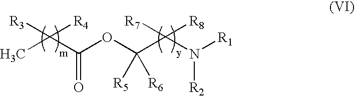

wherein m is an integer having a value in the range of about 5 to about 22, preferably, from about 5 to about 18; y is an integer having a value in the range of 0 to about 5; and R₁, R₂, R₃, R₄, R₅, R₆, and R₇ are members of the group consisting of hydrogen, C1 to C8 alkyl, and C6 to C8 aryl; and R₈ represents hydrogen, hydroxyl, C1 to C8 alkyl, or C6 to C8 aryl.

(N,N-disubstituted amino)alkanol alkanoates include C5 to C18 carboxylic acid esters, such as the compounds of the following formula (VI-1): 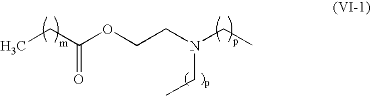
where m is an integer of from about 5 to about 21, preferably, from about 5 to about 16; and p is an integer of from 0 to about 3, preferably, 0 or 1, especially 0.

Exemplary specific alkyl alkanoate compounds of formula (VI) include 1-(N,N-dimethylamino)-2-propanol dodecanoate (DAIPD), 1-(N,N-dimethylamino)-2-propanol myristate (DAIPM), and 1-(N,N-dimethylamino)-2-propanol oleate (DAIPO).

Another class of penetration enhancers of type (iv) include N-alkyl lactams, such as those disclosed in, for example, U.S. Pat. Nos. 4,316,893 and 4,424,210, the disclosures of which are incorporated herein, in their entirety, by reference thereto; and N-alkylazacycloheptanes, such as those disclosed in, for example, U.S. Pat. No. 5,204,339, the disclosure of which is incorporated herein, in its entirety, by reference thereto.

The N-alkyl lactams include, for example, compounds of the following formula (VII): 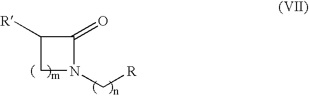
m is an integer of 3 to 7, n is 0 or an integer of 1 to 17, except that when m is 3, n is from 7 to 17, and R is preferably methyl.

A class of lactams represented by the following formula (VII-1) may also be used as SPE's: 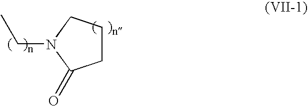
where n=0 or 1, and n″=0, 1 or 2.

Examples of compounds of formula (VII) include:
1-n-hexylazacyclopentan-2-one
1-n-heptaylazacyclopentan-2-one
1-n-octylazacyclopentan-2-one
1-n-nonylazacyclopentan-2-one
1-decylazacyclopentan-2-one
1-n-dodecylazacyclopentan-2-one
1-methylazacycloheptan-2-one
1-n-propylazacycloheptan-2-one
1-n-butylazacycloheptan-2-one
1-n-octylazacycloheptan-2-one
1-phenylazacyclopentan-2-one
1-(2-chlorophenyl)azacyclopentan-2-one
1,3-bis-(1-azacyclopentan-2-onyl)propane.

Of these, 1-n-dodecyl-azacycloheptan-2-one, is commercially available under the tradename AZONE.

The N-alkylazacycloheptanes may be represented by the following formula (VIII): 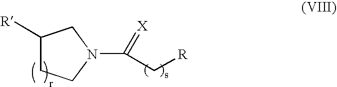
where X represents O or S, preferably O, R′ represents H or C1 to C4 alkyl; r is an integer of from 2 to 6, and s is 0 or an integer of 1 to 17.

Representative compounds of formula (VIII) include:
1-n-undecylformylazacycloheptane
1-n-decylformylazacycloheptane
1-n-octylformylazacycloheptane
1-n-nonylformylazacycloheptane
1-n-dodecylformylazacycloheptane
1-n-tetradecylformylazacycloheptane
1-n-hexadecylformylazacycloheptane
1-n-pentadecylformylazacycloheptane
1-n-heptadecylformylazacycloheptane
1-(16-methylhexadecyl)formylazacycloheptane.

When one or more SPE compounds are used in addition to a compound of formula (I) and/or formula (II), such as one or more of the types (i)-(iv) above, the amount of such other SPE compound will usually be within the range of from about 0.1 wt. % to about 10 wt. %, based on the total formula. The total amount of SPE compounds may be within the range of from about 0.1 to about 20 wt. %.

Active Agents

As used herein, the term “active agent” means any chemical or biological material suitable for administration, that produces a desired biological, pharmacological, or physiological effect in an animal or plant to which the agent is administered. Such effects may include, but are not limited to (1) having a prophylactic effect on the animal or plant, such as preventing an undesired biological effect, for example, as in preventing an infection; (2) alleviating a condition caused by a disease of the animal or plant, for example, alleviating pain or inflammation caused as a result of disease; and/or (3) either alleviating, reducing, or completely eliminating a disease from the animal or plant. The effect may be local, such as providing for a local anesthetic effect, or it may be systemic. Active agents are present in a pharmaceutically effective amount. The term “animal” as use herein is understood to also include human beings as well as other mammals, such as, for example, canine, feline, equine, bovine, porcine, ovine, including domestic animals, for example, cats and dogs.

Active agents that may be used in the compositions of the present invention include any locally or systemically active agents which are compatible with the compositions of the present invention and which can be delivered through the skin or other membrane to achieve a desired effect. In addition to biologically and pharmaceutically active agents, the SPE compounds of formulas (I) and (II) according to the present invention may also be used to facilitate delivery into the skin or other membranes of other active agents, such as cosmetic agents.

Active agents useful in embodiments of the present invention may be: polar, nonpolar, ionic, non-ionic, hydrophilic, lipophilic, water soluble or water insoluble.

Representative active agents (grouped by therapeutic class) include but are not limited to:

bronchodilators, such as, sodium cromoglycate, salbutamol or theophylline;

diuretic agents, such as, furosemide or hydrochlorothiazide;

antibacterial agents, such as, a penicillin, a cephalosporine, tetracycline, oxytetracycline, chlortetracycline or chloramphenicol;

antifungal agents, such as, amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione;

antiacne agents, such as, erythromycin;

sedatives or tranquillizers, such as, pentobarbital, secobarbital or codeine;

psychostimulants, such as, 3-(2-aminopropyl)indole acetate or 3-(2-aminobutyl)indole acetate;

anxiolytic agents, such as, diazepam, chlordiazepoxide, reserpine, chlorpromazine, buspirone hydrochloride or thiopropazate;

oestrogens, such as, oestradiol, oestriol, oestrone, ethinyloestradiol, mestranol, stilboestrol, dienoestrol, epioestriol, estropipate and zeranol;

hormonal drugs (hormones), such as, androgens, such as, for example, androstenediol and androisoxazole, testosterone, dihydrotestosterone, dehydroepiandrostenone; estrogens, such as, for example, 17 beta-estradiol, estradiol-3,17-diacetate, estradiol-3-acetate, estradiol-17-acetate, estradiol-3,17-valerate, estradiol-3-valerate, estradiol-17-valerate, ethinyl estradiol, estrone; progesterones, such as, for example, progesterone (preg-4-ene-3,20-dione), norethindrone, norgestrieone, norgestadienone, norgestrel, norgestimate, progestogenic acid, dihydroprogesterol, nomagesterol. Furthermore, in the above listed exemplary hormones, the testosterone hormone may be used in any of its usual forms, such as, for example, acetate, propionate, 17-beta-cyclopentanepropionate, enanthanate, isobutyrate, undeconate, and the like. Similarly, the estradiols may additionally be used in any of the known or newly developed forms, such as, for example, pivalate, propionate, cypionate, benzoate and other esters. Among these, especially preferred, based on the current level of knowledge in the pharmacological arts, are testosterone, progesterone and estradiol, in any of the salt or ester forms. More generally, however, any of the government approved hormones, such as listed in, for example, the most current edition of The Merck Index, may be advantageously used:

Other pharmacologically active agents include, for example,

ovulation inducers such as clomiphene;

antipyretic agents, such as, acetylsalicylic acid, salicylamide, sodium salicylate or methyl salicylate;

narcotic analgesics, such as, morphine or a major analgesic;

hypoglycaemiants, for example, a sulphonylureas such as glypizide, glyburic, chlorpropamide or insulin;

antispasmodic agents, such as, atropine or methscopolamine bromide;

antimalaria agents, such as, 4-aminoquinoline or 9-aminoquinoline;

beta-blockers, such as, metoprolol;

antiarthritic agents, such as, sulindac;

non-steroidal antiinflammatory drugs (NSAID), such as, heteroaryl acetic acids, such as, for example, tolmetin, diclofenac, ketorolac; arylpropionic acids, such as, for example, ibuprofen, naproxen, flurbiprofen, ketoprofen, fenoprofen, oxaprozin; anthranilic acids (fenamates), such as, for example, mefenamic acid, meclofenamic acid; enolic acids, such as, for example, oxicams (e.g., piroxicam, tenoxicam), pyrazolidinediones (e.g., phenylbutazone, oxyphenthatrazone); alkanones, such as, for example, nabumetone. Among these, especially preferred, based on the current level of knowledge in the pharmacological arts, are ibuprofen, diclofenac, ketorolac, naproxen, flurbiprofen, ketoprofen and piroxicam. More generally, however, any of the government approved NSAIDs, such as listed in, for example, the most current edition of The Merck Index, may be advantageously used.

Still other examples of pharmacologically active agents which may be transdermally delivered more efficiently according to the embodiments of the invention include the following:

anti-osteoporotic agents, such as, etidronate, or tiludronate;

skin bleaching agents, such as, ascorbic acid or hydroquinone;

vasodilators, such as, dipyridamole, the prostaglandins, trinitrine or isosorbide dinitrate;

prostaglandins, such as, alprostadil (PGE1), prostacyclin (PGI2), dinoprost (prostaglandin F2-alpha) and misoprostol;

other drugs useful in treating male or female sexual dysfunction such as papaverine, dioxyline, ethaverine, minoxidil, nitroglycerin, alpha blockers, nitric oxide donors;

corticosteroids, such as, betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate methylprednisolone, prednisolone, prednisolone 21-phosphate, prednisone, triamcinolone, triamcinolone acetonide;

steroidal agents, such as, cortodoxone, fluoracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol;

anti-hypertensive agents, such as, propanolol, prazosin, diltiazem or clonidine;

antiparkinsonian agents, such as, methyldopa or selegiline;

antimigraine agents, such as dihydroergotamine;

antiulcer agents, such as, cimetidine;

anticancer agents, such as, tamoxifen, cis-platin or the like;

nutritional agents, such as, vitamins, essential amino acids or essential fatty acids.

Other useful active agents according to the present invention may also include hydromorphone, hydroquinone, tentanyl, nalozone, nalbuphine, buprenorphine, methylphenidate, selegiline, pimozide, buspirone, oxybutynin, tacrolimus, mupirocin, bromocryptine, naproxen, diclofenac, ibuprofen, prostaglandin E1, testosterone, terbinafine or econazole.

As mentioned above, the compositions of the invention may optionally contain active agents formed of a combination of several medicinal substances selected from the groups listed above. It is also known that active agents, such as those mentioned above, may often have multiple biological or pharmacological effects.

The active agents may be present in the compositions in pharmacologically, pharmaceutically or cosmetically effective amounts and will depend on such factors as the disease or condition being treated, the age of the patient and other factors well understood by those skilled in the art. Generally, amounts of active agent may range from about 0.01 wt. % to about 15 wt. % relative to the weight of the total composition, such as from about 1 wt. % to about 15% wt, such as from about 0.5 wt. % to about 5 wt. %, or from about 1 wt. % to about 10 wt. %, or from about 1 wt. % to about 5 wt. %, for example, from about 1.0 wt. % or 1.5 wt. % to about 3.0 wt. % or 3.5 wt. % by weight of the composition.

Vehicles

The composition according to the invention may also comprise a solid, semi-solid or liquid pharmaceutically acceptable vehicle, to help the active agent and skin penetration enhancer to be conveyed to the skin or other membrane, such as the nasal or oral mucosa, at an appropriate concentration and amount. The nature of the vehicle will depend upon the method chosen for topical administration of the composition.

The selection of a vehicle for this purpose presents a wide range of possibilities depending on the required product form of the composition.

It should be explained that vehicles are compositions which may include diluents, dispersants, or solvents for the active agent and penetration enhancer which therefore ensure that they can be applied to and distributed evenly over an appropriate area of the skin. Compositions according to this invention can include water as a vehicle, and/or at least one pharmaceutically acceptable vehicle other than water.

Vehicles other than water that may be used in compositions according to the invention include solids or liquids such as emollients and moisturizers, solvents, humectants, thickeners, preservatives, colorants, fragrances, propellants and solid additives. Examples of types of such additives, which can be used singly or as mixtures, are as follows:

Representative emollients and moisturizers, include, for example, stearyl alcohol, glyceryl monoricinoleate, glyceryl monostearate, propane-1,2-diol, butane-1,3-diol, mink oil, cetyl alcohol, ispropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristoate.

Representative propellants include, for example, trichlorofluoromethane, dichlorodifluoromethane, dichlorotetrafluoroethane, monochlorodifluoromethane, trichlorotrifluoroethane, propane, butane, isobutane, carbon dioxide.

Representative solvents include, for example, lower alcohols, polyols, polyethers, oils, esters, alkyl ketones, and the like. For instance, mention may be made of ethyl alcohol, methylene chloride, isopropanol, castor oil, ethylene glycol monoethyl ether, diethylene glycol monobutyl ether, diethylene glycol monoethyl ether, dimethyl sulphoxide, dimethyl formamide, tetrahydrofuran. The solvent may be selected for its ability to dissolve the active agent, and the SPE compound or SPE compounds, and one of ordinary skill in the art would understand which solvents would be suitable for such purposes, or how to determine which solvents would be appropriate.

Representative humectants include, for example, glycerin, sorbitol, sodium 2-pyrrolidone-5-carboxylate, soluble collagen, gelatin, may be used in embodiments according to the invention.

Representative solid additives include, for example, chalk, talc, fullers earth, kaolin, starch, gums, colloidal silicon dioxide, tetra alkyl and/or trialkyl aryl ammonium smectites, chemically modified magnesium aluminium silicate, organically modified montmorillonite clay, hydrated aluminum silicate, fumed silica, carboxyvinyl polymers, hydroxyalkylated cellulosics, sodium carboxymethyl cellulose.

The amount of the vehicle can comprise the balance of the composition. Accordingly, the vehicle or vehicles may comprise up to about 99.9%, for example, from about 50 to about 99%, for example, from about 70 to about 95%, for example, from about 70 to about 99% by weight of the composition.

The above-described ingredients can be formulated with the skin penetration enhancer and active agent to form a composition suitable for topical application, including creams, lotions, ointments, gels, sprays, aerosols, and the like. In one embodiment, the active agent and skin penetration enhancer are dispersed within cream bases or ointment bases to form a cream or ointment.

Topical carriers may include conventional emulsifiers or other excipients including alginates, glyceryl stearate, PEG-100 stearate, cetyl alcohol, propylparaben, butylparaben, sorbitols, polyethoxylated sorbitan, fatty esters (TWEENS), white soft paraffin (petrolatum), triethanolamine, aloe vera extract, lanolin, cocoa butter, and the like. Suitable topical carriers are well known to the skilled artisan.

Preparation and Administration

The compositions according to the invention are well suited for topical, e.g., transdermal administration and may be prepared, in a conventional manner, by mixing together the various constituents in the chosen proportions. Different active agents may yield different results with different skin penetration enhancers, solvent or carrier systems or other ingredients in the formulation and in light of the present disclosure, the skilled artisan would be able to select an appropriate enhancer with the appropriate system for a given active agent.

The compositions of the invention thus obtained may be applied by any means to a predetermined area of skin, for example to an area of between 10 and 100 cm², for example 50 cm².

When the pharmaceutical compositions of this invention are in the form of a lotion, cream, emulsion, gel, solution, ointment or similar composition, the compositions may be spread as a film over the selected area of skin and, to this end, do not necessarily require intermediate propellant gases. Alternatively, the topical transdermal composition may also be incorporated into a transdermal delivery device, e.g., a patch.

In another embodiment of the present invention, the compositions may be administered by direct spraying using a doser pump of a type which is known and marketed for use without the aid of a propellant. If so desired, the compositions of the invention may, however, be administered by spraying from a container fitted with a dose valve, additionally containing a compressed propellant gas such as those mentioned above.

### EXAMPLES

The following examples are given as particular embodiments of the invention and to demonstrate the practice and advantages thereof. It is understood that the examples are given by way of illustration only and are not intended to limit the specification or the claims that follow in any manner.

### Example 1

The following compositions were prepared as ethanolic solutions of the indicated active agents and skin penetration enhancing compounds, and subsequently tested for transdermal penetration.

TABLE 1

| | Sample # | | | | | | | | | | | | | | | | | |
|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 |
| Reagent: | % | % | % | % | % | % | % | % | % | % | % | % | % | % | % | % | % | % |
| Ibuprofen | | | | | | | | 5 | 5 | 5 | 5 | 5 | 5 | 5 | | | | |
| Testosterone | 1 | 1 | 1 | 1 | 1 | 1 | 1 | | | | | | | | | | | |
| PGE1 | | | | | | | | | | | | | | | 1 | 1 | 1 | 1 |
| Hydroquinone | | | | | | | | | | | | | | | | | | |
| Buspirone HCl | | | | | | | | | | | | | | | | | | |
| t-Butyl Laurate | 2 | 5 | 10 | | | | | 2 | 5 | 10 | | | | | 2 | 5 | 10 | |
| Lauryl Pivalate | | | | 2 | 5 | 10 | | | | | 2 | 5 | 10 | | | | | 2 |
| t-Butyl | | | | | | | | | | | | | | | | | | |
| Decanoate | | | | | | | | | | | | | | | | | | |
| Decel Pivalate | | | | | | | | | | | | | | | | | | |
| Tetradecyl | | | | | | | | | | | | | | | | | | |
| Pivalate | | | | | | | | | | | | | | | | | | |
| N-Decyl | | | | | | | | | | | | | | | | | | |
| Pivalamide | | | | | | | | | | | | | | | | | | |
| N-Dodecyl | | | | | | | | | | | | | | | | | | |
| Pivalamide | | | | | | | | | | | | | | | | | | |
| t-Butyl | | | | | | | | | | | | | | | | | | |
| Myristoate | | | | | | | | | | | | | | | | | | |
| EtOH | 97 | 94 | 89 | 97 | 94 | 89 | 99 | 93 | 90 | 85 | 93 | 90 | 85 | 95 | 97 | 94 | 89 | 97 |
| Water | | | | | | | | | | | | | | | | | | |
| Total | 100 | 100 | 100 | 100 | 100 | 100 | 100 | 100 | 100 | 100 | 100 | 100 | 100 | 100 | 100 | 100 | 100 | 100 |
| | Sample # | | | | | | | | | | | | | | | | | |
| | 19 | 20 | 21 | 22 | 23 | 24 | 25 | 26 | 27 | 28 | 29 | 30 | 31 | 32 | 33 | 34 | 35 | 36 |
| Reagent: | % | % | % | % | % | % | % | % | % | % | % | % | % | % | % | % | % | % |
| Ibuprofen | | | | 5 | 5 | | | | | | | | | 5 | 5 | 5 | 5 | |
| Testosterone | | | | | | 1 | | | | | | | | | | | | 1 |
| PGE1 | 1 | 1 | 1 | | | | 2 | 2 | 2 | 2 | | | | | | | | |
| Hydroquinone | | | | | | | | | | | 3 | 3 | 3 | | | | | |
| Buspirone HCl | | | | | | | | | | | | | | | | | | |
| t-Butyl Laurate | | | | | | | | | 10 | | | | 10 | | | | | |
| Lauryl Pivalate | 5 | 10 | | | | | | | | 10 | 10 | | | | | | | |
| t-Butyl | | | | 10 | | | 10 | | | | | | | | | | | |
| Decanoate | | | | | | | | | | | | | | | | | | |
| Decel Pivalate | | | | | | | | | | | | | | 10 | | | | 10 |
| Tetradecyl | | | | | | | | | | | | | | | 10 | | | |
| Pivalate | | | | | | | | | | | | | | | | | | |
| N-Decyl | | | | | | | | | | | | | | | | 10 | | |
| Pivalamide | | | | | | | | | | | | | | | | | | |
| N-Dodecyl | | | | | | | | | | | | | | | | | 10 | |
| Pivalamide | | | | | | | | | | | | | | | | | | |
| t-Butyl | | | | | 10 | 10 | | 10 | | | | | | | | | | |
| Myristoate | | | | | | | | | | | | | | | | | | |
| EtOH | 94 | 89 | 99 | 85 | 85 | 89 | 88 | 88 | 88 | 88 | 87 | 97 | 87 | 85 | 85 | 85 | 85 | 89 |
| Water | | | | | | | | | | | | | | | | | | |
| Total | 100 | 100 | 100 | 100 | 100 | 100 | 100 | 100 | 100 | 100 | 100 | 100 | 100 | 100 | 100 | 100 | 100 | 100 |
| | Sample # | | | | | | | | | | | | | | | | | |
| | 37* | 38 | 39 | 40 | 41 | 42 | 43 | 44 | 45 | 46 | 47 | 48 | 49 | 50 | 51 | 52 | 53 | 54 |
| Reagent: | % | % | % | % | % | % | % | % | % | % | % | % | % | % | % | % | % | % |
| Ibuprofen | | | | | | | | | | | | | | | | | | |
| Testosterone | 1 | 1 | 1 | 1 | | | | | | | | | | | | | | |
| PGE1 | | | | | 1 | 1 | 1 | 1 | 1 | 2 | 2 | 2 | 2 | | | | 1 | 1 |
| Hydroquinone | | | | | | | | | | | | | | | | | | |
| Buspirone HCl | | | | | | | | | | | | | | 10 | 10 | 10 | | |
| t-Butyl Laurate | | | | | | | | | | | | | | | | | | |
| Lauryl Pivalate | | | | | | | | | | | | | | 10 | | | | |
| t-Butyl | | | | 10 | | | | | | | | | | | 10 | | 10 | |
| Decanoate | | | | | | | | | | | | | | | | | | |
| Decel Pivalate | | | | | 10 | | | | | 10 | | | | | | | | |
| Tetradecyl | 10 | | | | | 10 | | | | | 10 | | | | | | | |
| Pivalate | | | | | | | | | | | | | | | | | | |
| N-Decyl | | 10 | | | | | 10 | | 10 | | | 10 | | | | | | |
| Pivalamide | | | | | | | | | | | | | | | | | | |
| N-Dodecyl | | | 10 | | | | | 10 | | | | | 10 | | | | | |
| Pivalamide | | | | | | | | | | | | | | | | | | |
| t-Butyl | | | | | | | | | | | | | | | | | | 10 |
| Myristoate | | | | | | | | | | | | | | | | | | |
| EtOH | 89 | 89 | 89 | 89 | 89 | 89 | 89 | 89 | 89 | 88 | 88 | 88 | 88 | 68 | 68 | 76.5 | 89 | 89 |
| Water | | | | | | | | | | | | | | 12 | 12 | 13.5 | | |
| Total | 100 | 100 | 100 | 100 | 100 | 100 | 100 | 100 | 100 | 100 | 100 | 100 | 100 | 100 | 100 | 100 | 100 | 100 |

Sample #

| | 55 | 56 | 57 | 58 | 59 | 60 | 61 | 62 | 63 | 64 | 65 | 66 |
|---|---|---|---|---|---|---|---|---|---|---|---|---|
| Reagent: | % | % | % | % | % | % | % | % | % | % | % | % |
| Ibuprofen | | | | | | | | | | 5 | 5 | |
| Testosterone | | | | | | | | | | | | |
| PGE1 | | | | | | | | | | | | |
| Hydroquinone | 3 | 3 | 3 | 3 | 3 | | | | | | | |
| Buspirone HCl | | | | | | 10 | 10 | 10 | 10 | | | 10 |
| t-Butyl Laurate | | | | | | | | | | | | 10 |
| Lauryl Pivalate | | | | | | | | | | | | |
| t-Butyl | | | | 10 | | | | | | 10 | | |
| Decanoate | | | | | | | | | | | | |
| Decel Pivalate | | | | | | | | | | | | |
| Tetradecyl | 10 | | | | | 10 | | | | | | |
| Pivalate | | | | | | | | | | | | |
| N-Decyl | | 10 | | | | | 10 | | | | | |
| Pivalamide | | | | | | | | | | | | |
| N-Dodecyl | | | 10 | | | | | 10 | | | | |
| Pivalamide | | | | | | | | | | | | |
| t-Butyl | | | | | 10 | | | | 10 | | 10 | |
| Myristoate | | | | | | | | | | | | |
| EtOH | 87 | 87 | 87 | 87 | 87 | 68 | 68 | 68 | 68 | 85 | 85 | 68 |
| Water | | | | | | 12 | 12 | 12 | 12 | | | 12 |
| Total | 100 | 100 | 100 | 100 | 100 | 100 | 100 | 100 | 100 | 100 | 100 | 100 |

*at pH ranges from approximately 5.5 to 6.5, these solutions were unstable.

### Example 2

The following experiments were conducted to measure the flux of various active agents across human skin in the presence of an skin penetration enhancer. Human cadaver skin was obtained from AATB accredited tissue banks. The tissue was recovered within 15 hours of death or within 24 hours if the body was refrigerated, and prepared for these experiments using standard techniques.

Percutaneous absorption was measured using horizontal glass diffusion cells consisting of a donor and a receptor compartment (Franz-type diffusion cells, or static cells, supplied by Crown Glass Company of Somerville, N.J., U.S.A). The area available for diffusion was 0.635 cm2 and the receptor compartment volume was 5.5 mL. The receptor chamber was filled, so the liquid interfaced with the skin membrane, with approximately 5 mL buffered saline containing Volpo 20 in an amount sufficient to dissolve the active agent, and allowed to equilibrate to the correct temperature. Temperature of the skin surface was maintained at 32° C. throughout the experiment by placing diffusion cells into dry block heater set on 37° C. The receptor compartment contents were continuously agitated by small PTFE-coated magnetic stirring bars.

Formulations were then applied using a micropipette. The pipette was weighed before and after application and the amounts applied were recorded. Following application of the products, the entire receptor phase was removed at regular time intervals using a syringe. Following the final receptor phase sample, the residual drug remaining on the surface of the skin was determined.

Analytical determinations were made by high performance liquid chromatography (HPLC) using an Agilent HPLC system equipped with a variable wavelength detector, column oven, in-line degasser and autosampler.

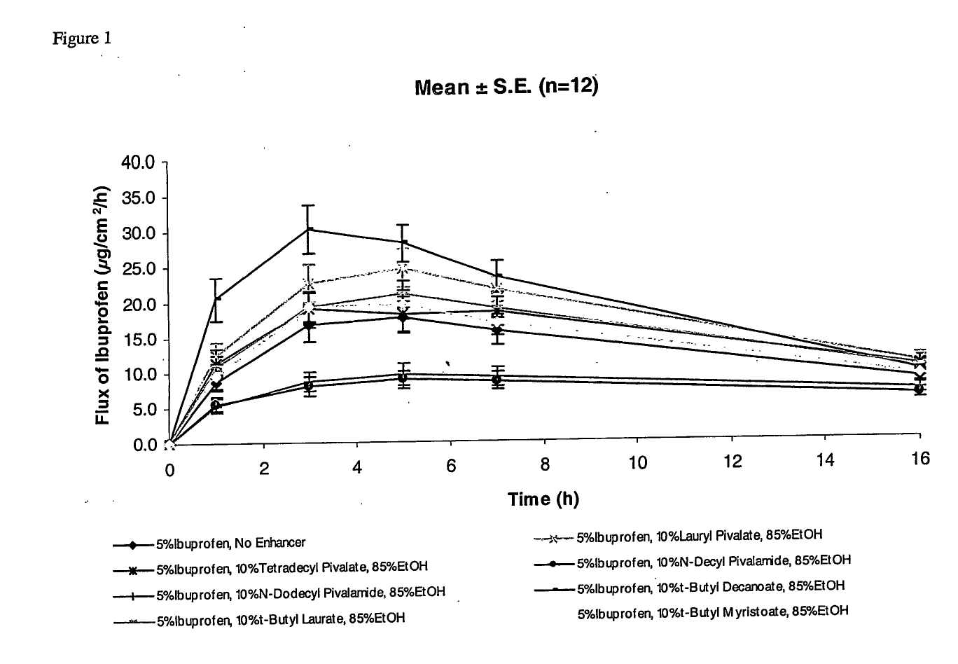

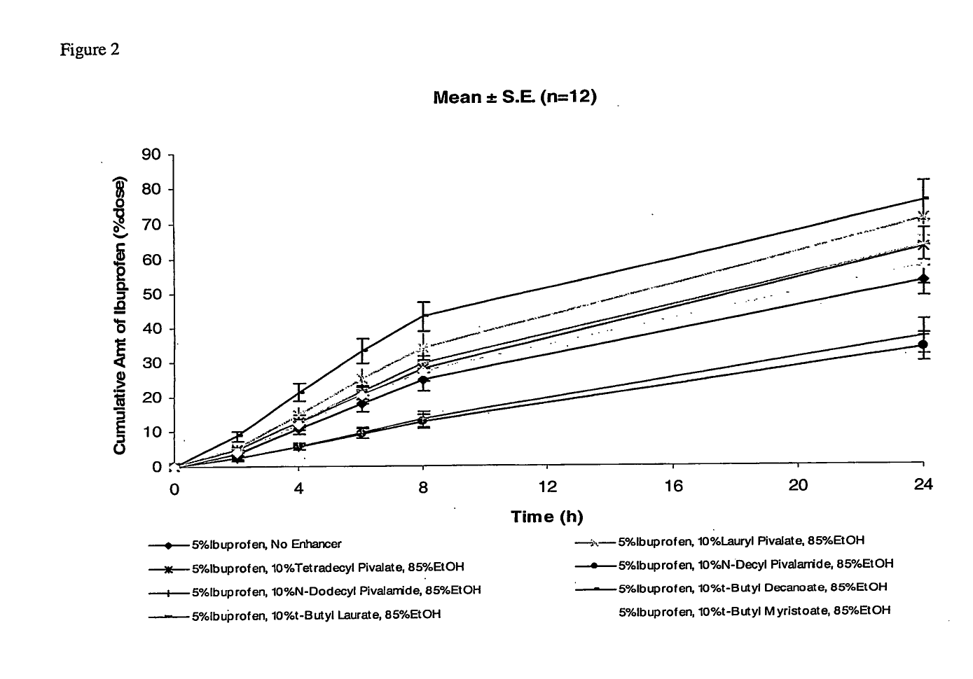

Data representing both the flux and cumulative transfer of active agent are reported graphically in the appended Figures. The number of time the experiment was repeated is expressed by the number “n” in the figures. As can be seen from these Figures, the skin penetration enhancers of formulas IA and IB increased the penetration of active agents through human skin with all the chosen active agents and enhancers, except the case where Ibuprofen was used with N-decyl pivalamide or N-dodecyl pivalamide (FIGS. 1 and 2). In this case, the rate of administration of Ibuprofen may be modified by its combination with N-decyl pivalamide or N-dodecyl pivalamide. These two cases are believed to be anomalous, as the amide enhancers of the present invention work well with hydrophobic systems, and were noted to provide enhancement with PGE-1 or testosterone systems, for example, as seen in the Figures.

Having described specific embodiments of the present invention, it will be understood that many modifications thereof will readily appear or may be suggested to those skilled in the art, and it is intended therefore that this invention is limited only by the spirit and scope of the following claims.

What is claimed is:

1. A pharmaceutical composition comprising:
a) at least one active agent; and
b) a skin penetration enhancer represented by the following Formula I: 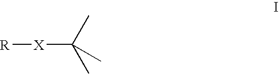
wherein: R represents a linear, saturated or unsaturated, substituted or unsubstituted hydrocarbyl radical; and
X represents a —(CO)O—, —O(CO)—, C═O, CONH, O, NHCONH, S, or S═O radical.

2. A pharmaceutical composition comprising:
a) at least one active agent; and
b) a skin penetration enhancer represented by the Formulas IA or IB 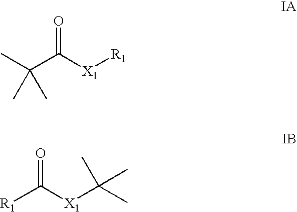
wherein:
R₁ represents a linear or branched, saturated or unsaturated, substituted or unsubstituted hydrocarbyl radical; and
X₁ is either an oxygen atom or an NH radical.

3. The pharmaceutical composition according to claim 2, wherein the skin penetration enhancer comprises a compound of Formula IA.

4. The pharmaceutical composition according to claim 2, wherein the skin penetration enhancer comprises a compound of Formula IB.

5. The pharmaceutical composition according to claim 2, wherein R₁ represents a linear C6-C20 alkyl radical.

6. The pharmaceutical composition according to claim 2, wherein R₁ represents a linear C6-C16 alkyl radical.

7. The pharmaceutical composition according to claim 2, wherein R₁ represents a linear C6-C14 alkyl radical.

8. The pharmaceutical composition according to claim 2, wherein R₁ represents a linear C8-C14 alkyl radical.

9. The pharmaceutical composition according to claim 2, wherein R₁ represents an octyl, nonyl, decyl, undecyl, dodecyl, tridecyl, or tetradecyl radical.

10. The pharmaceutical composition according to claim 2, wherein R₁ represents an octyl, decyl, dodecyl or tetradecyl radical.

11. The pharmaceutical composition according to claim 2, wherein X₁ represents an oxygen atom.

12. The pharmaceutical composition according to claim 2, wherein X₁ represents an N—H radical.

13. The pharmaceutical composition according to claim 2, wherein the skin penetration enhancer of formula IA or IB is decyl pivalate, dodecyl pivalate, tetradecyl pivalate, N-decyl pivalamide, N-dodecyl pivalamide, tert-butyl decanoate, tert-butyl laurate, or tert-butyl myristate.

14. The pharmaceutical composition according to claim 2, further comprising a pharmaceutically acceptable vehicle.

15. The pharmaceutical composition of claim 14, which is in the form of a liquid.

16. The pharmaceutical composition of claim 14, which is in the form of a cream.

17. The pharmaceutical composition of claim 14, which is in the form of a lotion.

18. The pharmaceutical composition of claim 14, which is in the form of an ointment.

19. The pharmaceutical composition of claim 14, which is in the form of a gel.

20. The pharmaceutical composition of claim 14, which is in the form of a spray.

21. The pharmaceutical composition of claim 14, which is in the form of an aerosol.

22. The pharmaceutical composition of claim 2, wherein said active agent comprises at least one compound selected from amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, or pyrithione.

23. The pharmaceutical composition of claim 2, wherein said active agent comprises at least one compound selected from papaverine, dioxyline, ethaverine, minoxidil, or nitroglycerin.

24. The pharmaceutical composition of claim 2, wherein said active agent comprises at least one compound selected from alprostadil (PGE1), prostacyclin (PGI2), dinoprost (prostaglandin F2-alpha), or misoprostol.

25. The pharmaceutical composition of claim 2, wherein said active agent comprises at least one compound selected from tolmetin, diclofenac, ketorolac, ibuprofen, naproxen, flurbiprofen, ketoprofen, fenoprofen, oxaprozin, mefenamic acid, meclofenamic acid, fhilenamic acid, piroxicam, tenoxicam, phenylbutazone, oxyphenthatrazone, nabumetone.

26. A pharmaceutical composition according to claim 2, comprising:
a) from about 1 wt. % to about 15 wt. % buspirone hydrochloride; and
b) a penetration enhancing amount of a skin penetration enhancer represented by the Formulas IA or IB 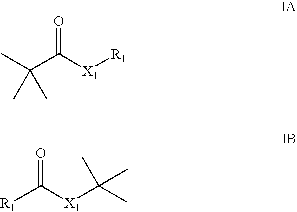
wherein:
R₁ represents a linear or branched, saturated or unsaturated, substituted or unsubstituted hydrocarbyl radical; and
X₁ is either an oxygen atom or an NH radical.

27. A pharmaceutical composition according to claim 2, comprising:
a) from about 1 wt. % to about 10 wt. % ibuprofen; and
b) a penetration enhancing amount of a skin penetration enhancer represented by the Formulas IA or IB 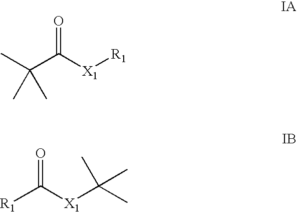
wherein:
R₁ represents a linear or branched, saturated or unsaturated, substituted or unsubstituted hydrocarbyl radical; and
X₁ is either an oxygen atom or an NH radical.

28. A pharmaceutical composition according to claim 2, comprising:
a) from about 0.5 wt. % to about 5 wt. % testosterone; and
b) a penetration enhancing amount of a skin penetration enhancer represented by the Formulas IA or IB 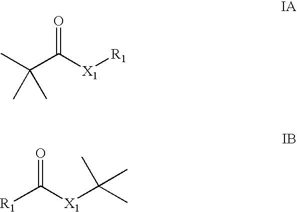
wherein:
R₁ represents a linear or branched, saturated or unsaturated, substituted or unsubstituted hydrocarbyl radical; and
X₁ is either an oxygen atom or an NH radical.

29. A pharmaceutical composition according to claim 2, comprising:
a) from about 0.5 wt. % to about 5 wt. % PGE-1; and
b) a penetration enhancing amount of a skin penetration enhancer represented by the Formulas IA or IB 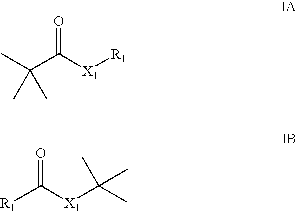
wherein:
R₁ represents a linear or branched, saturated or unsaturated, substituted or unsubstituted hydrocarbyl radical; and
X₁ is either an oxygen atom or an NH radical.

30. A pharmaceutical composition according to claim 2, comprising:
a) from about 1.5 wt. % to about 3.5 wt. % hydroquinone; and
b) a penetration enhancing amount of a skin penetration enhancer represented by the Formulas IA or IB 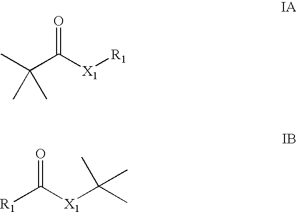
wherein:
R₁ represents a linear or branched, saturated or unsaturated, substituted or unsubstituted hydrocarbyl radical; and
X₁ is either an oxygen atom or an NH radical.

31. A method for forming a pharmaceutical or cosmetic composition comprising, mixing:
a) from about 1 wt. % to about 10 wt. % of an active agent; and
b) a skin penetration enhancer represented by the Formulas IA or IB 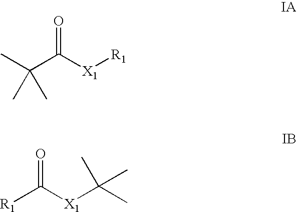
wherein:
R₁ represents a linear or branched, saturated or unsaturated, substituted or unsubstituted hydrocarbyl radical; and
X₁ is either an oxygen atom or an NH radical.

32. A method of administering an active agent to an animal in need thereof comprising topically applying to said animal a pharmaceutical composition comprising:
a) an active agent; and
b) a skin penetration enhancer represented by the Formulas IA or IB 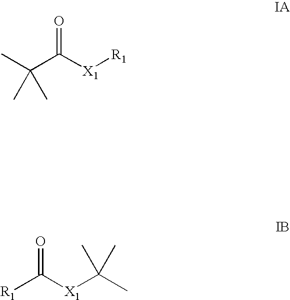
wherein:
R₁ represents a linear or branched, saturated or unsaturated, substituted or unsubstituted hydrocarbyl radical; and
X₁ is either an oxygen atom or an NH radical.

33. The method of claim 32, wherein said animal or plant is a human.

34. The method according to claim 32, wherein said active agent is a pharmacologically active agent.

35. The method according to claim 32, wherein said active agent is a cosmetic agent.

36. The method according to claim 32, wherein said active agent is a bronchodilator, diuretic agent, antibacterial agent, antifungal agent, antiacne agent, sedative, tranquilizer, psychostimulant, anxiolytic agent, oestrogen, hormone, ovulation inducer, antipyretic agent, narcotic analgesic, hypoglycaemiant, antispasmodic agent, antimalaria agent, beta-blocker, antiarthritic agent, non-steroidal antiinflammatory drug, anti-osteoporotic agent, skin bleaching agent, vasodilator, prostaglandin, corticosteroid, steroidal agent, anti-hypertensive agent, antiparkinsonian agent, antimigraine agent, antiulcer agent, anticancer agent or nutritional agent.

37. The method according to claim 32, wherein the active agent is at least one compound selected from the group consisting of: sodium cromoglycate, salbutamol, theophylline, furosemide, hydrochlorothiazide, penicillin, tetracycline, oxytetracycline, chlortetracycline, chloramphenicol, amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione, erythromycin, pentobarbital, secobarbital, codeine, 3-(2-aminopropyl)indole acetate, 3-(2-aminobutyl)indole acetate, diazepam, chlordiazepoxide, reserpine, chlorpromazine, buspirone hydrochloride thiopropazate, oestradiol, oestriol, oestrone, ethinyloestradiol, mestranol, stilboestrol, dienoestrol, epioestriol, estropipate, zeranol, androstenediol, androisoxazole, testosterone, dihydrotestosterone, dehydroepiandrostenone, 17 beta-estradiol, estradiol-3,17-diacetate, estradiol-3-acetate, estradiol-17-acetate, estradiol-3,17-valerate, estradiol-3-valerate, estradiol-17-valerate, ethinyl estradiol, estrone, progesterone, norethindrone, norgestrieone, norgestadienone, norgestrel, norgestimate, progestogenic acid, dihydroprogesterol, nomagesterol, clomiphene, acetylsalicylic acid, salicylamide, sodium salicylate, methyl salicylate, morphine, glypizide, glyburic, chlorpropamide, insulin, atropine, methscopolamine bromide, 4-aminoquinoline, 9-aminoquinoline, metoprolol, sulindac, tolmetin, diclofenac, ketorolac, ibuprofen, naproxen, flurbiprofen, ketoprofen, fenoprofen, oxaprozin, mefenamic acid, meclofenamic acid, piroxicam, tenoxicam, phenylbutazone, oxyphenthatrazone, nabumetone, etidronate, tiludronate, ascorbic acid, hydroquinone, dipyridamole, trinitrine, isosorbide dinitrate, misoprostol, papaverine, dioxyline, ethaverine, minoxidil, nitroglycerin, betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate methylprednisolone, prednisolone, prednisolone 21-phosphate, prednisone, triamcinolone, triamcinolone acetonide, cortodoxone, fluoracetonide, fludrocortisone, difluorsone diacetate, flurandrenolone acetonide, medrysone, amcinafel, amcinafide, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal, nivazol, propanolol, prazosin, diltiazem, clonidine, methyldopa, selegiline, dihydroergotamine, cimetidine, tamoxifen or cis-platin.

38. The method according to claim 32, wherein the active agent is at least one compound selected from hydromorphone, hydroquinone, tentanyl, nalozone, nalbuphine, buprenorphine, methylphenidate, selegiline, pimozide, buspirone, oxybutynin, tacrolimus, mupirocin, bromocryptine, naproxen, diclofenac, ibuprofen, prostaglandin E1, testosterone, terbinafine or econazole.
